(19) United States
(12) Patent Application Publication
(10) Pub. No.: US 2018/0360100 A1
(43) Pub. Date: Dec. 20, 2018

(54) Unexpectedly Enhanced Pigments Compositions and Processes

(71) Applicant: SCIENTIFIC HOLDINGS, LLC, Monrovia, CA (US)

(72) Inventors: Jeffrey Charles Raber, Pasadena, CA (US); Braden Doane, Bellevue, WA (US); Bradley J. Douglass, Seattle, WA (US)

(21) Appl. No.: 16/007,936

(22) Filed: Jun. 13, 2018

Related U.S. Application Data
(60) Provisional application No. 62/518,703, filed on Jun. 13, 2017.

(51) Int. Cl. A24B 15/16; A23L 2/58; A23G 3/48; A23L 5/43; A24B 15/34; A24F 47/00; A24B 15/40; A61K 8/92; A61K 8/31; A61K 8/35; A61K 8/58; A61Q 19/00

(52) U.S. Cl. CPC A24B 15/167; A23L 2/58; A23G 3/48; A23L 5/43; A24B 15/345; A24F 47/008; A23V 2002/00; A61K 8/922; A61K 8/31; A61K 8/35; A61K 8/58; A61Q 19/007; A24B 15/403

(57) ABSTRACT

Better than expected or empirically predicted odorant blends which contain one or more pigments, where the pigment influences the perceived identity of the odorants origin, such as an origin that is apple, lime, or grape, or where the pigment influences the preference for or nature of the perceived aroma, or influences an odor-associated flavor that may be evoked by the odorant, such as that of fruitiness, sweetness, sourness, or bitterness.

This application claims the benefit of and priority to U.S. Provisional Patent Application Ser. No. 62/518,703, filed Jun. 13, 2017, the content of which is incorporated herein by reference herein in its entirety. The disclosure relates to consumables such as electronic cigarettes, candles, incense, perfumes, beverages, and snack foods. The disclosure provides colorants and odorants, and formulations thereof, where the colorant influences the perceived odor.

### BACKGROUND OF THE DISCLOSURE

The color of a food and colorings added to a food are visual cues on taste and flavor. Greater color intensity can influence a subject's rating of a substance as having a greater intensity flavor, and can also influence identification of the flavor. Color can also tip the balance between two different perceptions of odor or flavor, for example, the perceptions of sweet versus sour. Odors can modify behavior, generate emotions, and evoke memories. Color can influence odor perception as shown, for example, by aroma determination tests of wine (see, e.g., Morrot G et al (2001) Brain and Language. 79:309-320). Regarding odor itself, odor testing can result in experimental data taking the form of reports of pleasantness, unpleasantness, intensity, and duration of memory of a given odor (see, e.g., Richardson J et al (1989) Psychological Bulletin. 105:352-360). A variety of tests are available for assessing the influence of color on the perceptions of a given liquid, for example, where color influences the perception of “refreshingness” or the perception of “thirst-quenchability” (see, e.g., Zellner D A et al (2003) Am. J. Psychol. 116:633-647). Color can be a more influential cue than taste in the consumers' perception of flavor and, in this way, a coloring can contribute to the marketing of a given product (see, e.g., Tom G et al (1987) Consumer Marketing, 4:23-27).

Food color influences perceived flavor intensity and identity. An example is oenophiles given white wine colored red who described it with red wine flavor descriptors. A cause of decreased food intake that occurs with advancing age is loss in taste and smell acuity. The present disclosure provides compositions for use in flavor-amplified foods to improve nutritional status in elderly persons. Moreover, the present disclosure provides cannabis compositions, optionally with flavorings and colorings, to improve appetite.

The present disclosure provides compositions and formulations where a coloring is included, and where the particular color influenced the subject towards certain odor-perceptions and also influenced the same subject away from other odor-perceptions.

### SUMMARY OF THE DISCLOSURE

Briefly stated, the present disclosure provides a formulation comprising a coloring and an odorant, wherein the odorant provides a perceivable odor to the formulation, and wherein the coloring provides a perceivable color to the formulation. Also provided, is the above formulation, wherein in the coloring in pure form does not have a perceivable odor, and wherein the odorant in purified form does not have a perceivable color.

In another aspect, what is provided is the above wherein the perceivable odor is orange, lemon, lime, or berry. Moreover, what is contemplated is the above formulation, wherein, the coloring is orange, yellow, green or blue. In yet another embodiment, what is provided is the above formulation, that at room temperature and atmospheric pressure is one of a liquid, slurry, paste, oil, powdered solid, a non-powdered solid, or any combination thereof, In yet another aspect, what is set forth is the above formulation, wherein the odorant in purified form has a first odor, wherein the odorant in purified form mixed with a coloring in purified form has a second odor, and wherein the first odor is more intense than the second odor as determinable with perception tests with human subjects.

In yet another aspect, what is provided is the above formulation, wherein the odorant in purified form has a first odor, wherein the odorant in purified form mixed with a coloring in purified form has a second odor, and wherein, the first odor is less intense than the second odor as determinable with perception tests with human subjects. Moreover, what is embraced is the above formulation, wherein the odorant in purified form has first odor that is perceivable as smelling like a first kind of fruit, wherein the odorant in purified form mixed with a coloring in purified form has a second odor that is perceivable as smelling like a second kind of fruit.

In yet another aspect, what is provided is the above formulation, that is capable of use in an electronic cigarette, wherein the electronic cigarette is capable in use of emitting a smoke, and wherein the coloring is capable of being perceived as having a color in the smoke emitted by the electronic cigarette.

What is further embraced, is the above formulation that is capable of use in a beverage for human consumption. Additionally provided is the above that is capable of use in one or more of a skin lotion, perfume, cosmetic, for human consumption. In yet another embodiment, what is provided is the above formulation, that is capable of use in a confection, cake frosting, or candy, for human consumption, and the above formulation that comprises at least one plant pigment and at least one cannabinoid, and the above formulation that comprises at least one plant pigment and at least one terpene, and the above formulation that comprises at least one non-plant pigment and at least one cannabinoid, and the above formulation that comprises at least one non-plant pigment and at least one terpene.

What is provided is the above formulation that comprises D-Limonene, beta-caryophyllene, beta-myrcene, linalool, beta-pinene, alpha-humulene, alpha-bisabolol, alpha-pinene, terpinolene, but not any caryophyllene-oxide, optionally with orange pigment (Tangie). Provided is the above formulation, comprising orange pigment, wherein the orange pigment acts by synesthesia to increase preference by a human subject of one or both of the odor or flavor of Tangie.

What is embraced is the above formulation that comprises D-Limonene, beta-pinene, beta-caryophyllene, beta-myrcene, linalool, alpha-humulene, alpha-bisabolol, alpha-pinene, terpinolene, but not any caryophyllene-oxide, optionally with yellow pigment (Lemon Kush). What is further encompassed is the above formulation, comprising yellow pigment, wherein the yellow pigment acts by synesthesia to increase preference by a human subjects of one or both of the odor or flavor of Lemon Kush.

What is embraced is the above formulation that comprises D-Limonene, beta-myrcene, alpha-pinene, beta-caryophyllene, beta-pinene, alpha-humulene, linalool, alpha-bisabolol, caryophyllene-oxide, terpinolene, optionally with green pigment (Lime Dream). What is encompassed is the above formulation, comprising green pigment, wherein the green pigment acts by synesthesia to increase preference by a human subject of one or both of the odor or flavor of Lime Dream. What is provided is the above formulation, that comprises Beta-myrcene, beta-caryophyllene, D-Limonene, alpha-pinene, alpha-humulene, beta-pinene, linalool, alpha-bisabolol, terpinolene, caryophyllene-oxide, terpinolene, optionally with blue pigment (Berry Cush). What is encompassed is the above formulation, comprising blue pigment, wherein the blue pigment, acts by synesthesia to increase preference, by a human subject of one or both of the odor or flavor of Berry Cush.

The present disclosure provides a formulation for an electronic cigarette comprising 0.05%-5% guaiazulene, about 10% terpenes, about 65% phytol, and about 24.5% cannabinoids, wherein the perception of grape odor is greater than that for a formulation comprising 0-0.03% guaiazulene, about 10% terpenes, about 65% phytol, and about 24.5% cannabinoids. Also, what is provided is: (1) The above formulation with 0% guaiazulene, (2) The above formulation with 0.01% guaiazulene, (3) The above formulation with 0.02% guaiazulene, (4) The above formulation with 0.05% guaiazulene, (5) The above formulation with 0.1% guaiazulene, (6) The above formulation with 0.5% guaiazulene, (7) The above formulation with 2.0% guaiazulene, (8) The above formulation with 5.0% guaiazulene, (9) The above formulation with 10% guaiazulene. What can be excluded is any one or more of the above embodiments.

Moreover, the present disclosure also provides, a formulation for an electronic cigarette comprising 0.05%-5% of one or more azulene compounds, about 10% terpenes, about 65% phytol, and about 24.5% cannabinoids, wherein the perception of grape odor is greater than that for a formulation comprising 0-0.03% guaiazulene, about 10% terpenes, about 65% phytol, and about 24.5% cannabinoids.

In another aspect, what is provided is a formulation for an electronic cigarette comprising 0.05%-5% guaiazulene, about 10% terpenes, about 65% phytol, and about 24.5% cannabinoids, wherein the perception of grape odor is greater than that for a formulation comprising 0-0.03% guaiazulene, about 10% terpenes, about 65% phytol, and about 24.5% cannabinoids.

Also provided is a formulation for an electronic cigarette comprising 0.05%-5% guaiazulene, about 10% terpenes, about 65% phytol, and about 24.5% cannabinoids, wherein the perception of grape flavor is greater than that for a formulation comprising 0-0.03% guaiazulene, about 10% terpenes, about 65% phytol, and about 24.5% cannabinoids.

Moreover, what is contemplated is a for an electronic cigarette, comprising 0.01%-0.05% chamazulene, about 5% terpenes, and about 94.99% cannabinoids, wherein the perception of grape odor is greater than that for a formulation comprising 0-0.005% chamazulene, about 5% terpenes, and about 94.99% cannabinoids.

Moreover, what is embraced is a formulation for an electronic cigarette, comprising 0.01%- 0.05% chamazulene, about 5% terpenes, and about 94.99% cannabinoids, wherein the perception of grape flavor is greater than that for a formulation comprising 0-0.005% chamazulene, about 5% terpenes, and about 94.99% cannabinoids.

### DETAILED DESCRIPTION

As used herein, including the appended claims, the singular forms of words such as “a,” “an,” and “the” include their corresponding plural references unless the context clearly dictates otherwise. Al references cited herein are incorporated by reference to the same extent as if each individual publication, patent, and published patent application, as well as figures and drawings in said publications and patent, documents, was specifically and individually indicated to be incorporated by reference.

Expressly incorporated by reference are all serial numbers, patents, granted and issued overseas: Ser. No. 14/467,565 filed Aug. 25, 2014; PCT/US2014/056249 filed Sep. 18, 2014; U.S. Letters U.S. Pat. No. 9,732,009, issued Aug. 15, 2017; PCT2014/064860 filed Nov. 10, 2014; Ser. No. 15/014,716 filed Feb. 3, 2016; Ser. No. 15/097,197 filed Apr. 12, 2016; PCT/US16/44929 filed Jul. 30, 2016; Ser. No. 15/250,475 filed Aug. 29, 2016; 62/518,703 filed Jun. 13, 2017; Ser. No. 15/676,744 filed Aug. 14, 2017; 62/553,817 filed Sep. 2, 2017; 62/556,418 filed Sep. 9, 2017; Ser. No. 15/826,203 filed Nov. 29, 2017; PCT/US15/55272 filed Oct. 13, 2015; Ser. No. 14/882,018 filed Oct. 13, 2015; AU 2014 323 509 filed Sep. 18, 2014; CA 2,923,091 filed Sep. 18, 2014; IL Patent No. 244399, granted May 29, 2017; IN 201617007409 filed Sep. 18, 2014; RU 2016107794 filed Sep. 18, 2014; ES P201690015 filed Sep. 18, 2014; IL Patent No. 245469 granted May 29, 2017; IN 201617015721 filed Nov. 10, 2014; RU 2016117582 filed Nov. 10, 2014; ES Patent No. 2576464 issued May 19, 2017; AU 2015 333 703 filed Oct. 13, 2015; IL 251660 filed Oct. 13, 2015; IN 201717013784 filed Oct. 13, 2015; JP 2017-519651 filed Oct. 13, 2015; RU 2017112763 filed Oct. 13, 2015; ES 201790019 filed Oct. 13, 2015; UK 1707675.3 filed Oct. 13, 2015; CZ PV2017-259 filed Oct. 13, 2015; CA 2,994,266 filed Jul. 30, 2016; IL 257268 filed Jul. 30, 2016; Ser. No. 15/749,358 filed Jan. 31, 2018; AU 2018 201 704 filed Mar. 9, 2018.

The term “coloring” refers to a physical entity such as a chemical, molecule, metal ion, complex of metal ion and a molecule, composition comprising one or more of the above, composition consisting of one or more of the above, and the like, where the “coloring” is perceivable as having a color. Without implying any limitation, in some embodiments, a “coloring” in a relatively pure form or in a pure form, is perceivable as having a color, while in other embodiment., the “coloring” is perceivable has having a color only when subject to a specific environmental condition, such as an acid pH or a neutral pH.

Odorants, carriers, diluting oils such as dipropylene glycol, coconut oil, and glucam P-20, propylene glycol, triacetin, vegetable glycerin are available from, e.g., Perfumers Apprentice (Scotts Valley, Calif.). Colorings and pigments are available from, e.g., American Colors, Inc., Sandusky, Ohio. Natural aroma chemicals, synthetic aroma chemicals, and essential oils and extracts are available from, for example, Vigon International, East Stroudsburg, Pa.. Violet-colored azulene compounds are disclosed by, e.g., Hayashi (1968) Bulletin of the Chemical Society of Japan. 41:2182-2183.

Standard color charts are available, for example, from Long J (2011) The New Munsell Student Color Set, 3rd ed. Fairchild Books; Centore P (2015) Controlling Colour With the Munsell System. ISBN-10:1632270129. The Munsell System is used in psychology experiments (see, e.g., Gilbert A N et al. (1006) Cross-modal correspondence between vision and olfaction: the color of smells. Am. J. Psychology. 109:335-351).

Other standard color charts include: ROL Colors. The RAL system is a Central European Color Standard, and RAL color charts are available from Color Harzen. General chemical reagents, as well as colorings, solvents, and surfactants are available (Sigma Aldrich, St. Louis, Mo.; Fischer Chemicals, Fair Lawn, N.J.; Ceriliiant, Round Rock, Tex.; Promochem, Molsheim, France, Cayman Chemical Co., Ann Arbor, Mich.). Useful labels include 33P, 35S, 14C, 3H, stable isotopes, fluorescent dyes, or fluorettes (see, e.g., Rozinov and Nolan (1998) Chem. Biol. 5:713-728). Terpenes, cannabinoids, formulations, and methods for preparation and analysis are available from US2015/0080265 of Elzinga and Raber, US2015/0152018 of Raber and Elzinga, 2017/0021029 of Raber et al, and 2016/0151328 of Doane and Raber, each of which is incorporated herein in its entirety.

The present disclosure provides compositions, reagents, and combinations of reagents, that act by way of synesthesia. Synesthesia encompasses, but is not limited to, a phenomenon where a stimulus in one sensory mode is interpreted in terms of some other sensory mode. For example, colors can bias odor judgments. Odor judgments include, and are not limited to, accuracy in odor identification. A synesthesia questionnaire can take the form of a paper that invokes a mood, such as angry, tired, proud, peaceful, surprised, excited, joyful, and so on, and where the subjected reports the evoked mood (e.g., yellow perceived as joyful, red as surprised, blue as peaceful). Also, the subject can be presented with an odor sample, and be asked to report the evoked color. Fragrances of the present disclosure can be dissolved in diethyl phthalate, where odor chemicals are present as, e.g., 0.1% to 5% to 40% and so on. Alternatively, the odor chemical can be used at 100% strength. See, Gilbert A N et al (1996.) Am. J. Psychology. 109:335-351; Rader and Tellegen 1987) J. Personality and Social Psychology. 52:981-987. In another aspect, the present disclosure can exclude compositions, reagents, methods, or publications, that do not act by way of synesthesia or that are not disclosed as acting by way of synesthesia.

The present disclosure provides compositions, formulations, solutions, and the like, capable of use with an electronic cigarette (e-cigarette). Apparatus and formulations for e-cigarettes are available. See, U.S. Pat. No. 9,254,002 of Chong and U.S. Pat. No. 8,997,753 of Li, which are incorporated herein in their entirety. In embodiments, the compositions, formulations, and solutions of the present disclosure are capable of being converted to an aerosol, and in other embodiments, take the form of an aerosol.

The reagents and compositions of the present disclosure can be used in combination with, or can be contained within, one or more of these devices. The devices are, a moist towel in a tearable aluminum foil envelope, scented soft paper tissue, e.g., Kleenex®, a cartridge that goes inside a bong, or water pipe, or hookah, where the composition supplements the flavor or medicinal effects of the marijuana smoke, an inhaler for people with asthma, where your composition is mixed with and supplements the anti-inflammatory drugs that are in the inhaler, coaled porous particles, coated non-porous particles, where the particles have terpenes and cannabinoids soaked into the particles, mixed in a hangover treatment (not necessary medicinal), as part of a composition with capsaicins (chili pepper chemical) for use in treating pain, for topical application, for arthritis pain or as part of an existing arthritis pain topical cream, lip balm, as a component of an oil-based cosmetic for skin. The present disclosure can also exclude one or more of the above embodiments, for example, it can exclude devices comprising a towel, soft paper tissue, a cartridge, a coated porous particle, a coated non-porous particle, capsaicins, lip balm, topical cream, and the like.

DABS. Dabs are a means for consuming cannabis concentrates via vaporization and inhalation. A dab usually refers to a dose of concentrate that is heated on a hot surface, usually a nail, and then inhaled through a dab rig. Dabs are concentrated doses of cannabis that are made by extracting THC and other cannabinoids using a solvent such as butane or carbon dioxide, resulting in sticky oils also commonly referred to as wax, shatter, budder, and butane hash oil (BHO). While it is possible to extract non-psychoactive compounds like CBD, THC is what is behind the potent effects of dabs, increasing efficiency in administration of medications. Terpenes or the aromatic oils that give cannabis flavor, can also be extracted. The present disclosure provides methods of use, methods of manufacture, and compositions and reagents for use with administration by way of dabs.

ECIG COMPONENTS. The present disclosure provides devices and methods for storing our compositions inside of a vapor cartridge (ecig component) or other device or object. Devices and objects may be color coded to indicate flavor suggestions, and optionally also to indicate if it contains THC or CBD (or other cannabinoid). A non-limiting physical structure of the ecig includes: (1) Chip/microcontroller for voltage regulation, run lights or vibration, flow cell monitoring; (2) LEDs/vibrating motor for visual and tactile feedback; (3) Battery to supply power; (4) Chamber to hold material to be vaporized. The chamber can be of many shapes and materials; (5) Mouthpiece; (6) Heating element to vaporize the material. Can be made in many shapes and material; (7) 510 thread is the standardized linkage between cartridges of material and the battery/electronics; (8) The chamber part (also known as a cartridge) is where the composition is held (compositions could be placed in vapor cartridges that are transparent in their construction to allow seeing the composition). In a vapor cartridge it would be intended to smelled by the nose during exhale, and tasted by the tongue on inhalation. In other uses, such as a dab, the user would intend to smell before inhalation and smell during exhalation too, so both. If one has single or both depends on how it is delivered really. The present disclosure provides methods of use, methods of manufacture, and compositions and reagents for use with administration by way of an ecig.

Methods for conducting odorant perception tests are available. In one type of test subjects are instructed to describe the odor of various fruit solutions, some of which are inappropriately colored. Then, subjects are presented with a series of fruit solutions which vary in odor and color intensity and then asked to rank them in order of odor strength (Blackwell L (1995) Nutrition and Food Science. 95:24-28). In another type of test, an odorant is presented in one of 16 different dilutions. The odorant may or may not be colored. Also, a picture or photograph of a possible source of the odorant may or may not be shown to the subject, for example, a picture of a ham, cookie, or melon. The subject then identifies the odorant (Gudziol V and Hummel T (2009) Arch. Otoloaryngol. Head Neck Surg. 135:1431-1435). In other types of tests, stimulant delivery occurred via squeezable polyethylene bottles that could deliver odorized air directly into the nostril, or alternatively, into air located below the nostrils (Rabin M D and Cain W S (1986) Perception Psychophysics. 39:281-286).

Entourage compounds and entourage effects are encompassed by the present disclosure. The methods, compositions, reagents, and devices of the present disclosure encompass entourage effects of cannabis and cannabinoids, including entourage effects provided by terpenes. Interactions between phytocannabinoids and terpenoid compounds can act synergistically for treating pain, inflammation, depression, anxiety, and addiction. Ben-Shabat et al provide an example of an entourage effect. 2-Arachidonoyl-glycerol is an endogenous ligand for both cannabinoid receptors, CB1 and CB2. The biological activity of 2-arachidonoyl-glycerol can be increased by related 2-acyl-glycerols, which alone show little or no activity. This effect (entourage effect) enhances activity of cannabinoids. 2-linoleoyl-glycerol and 2-palmitoyl-glycerol are two esters do not bind to cannabinoid receptors, nor do they inhibit adenylyl cyclase via either CB1 or CB2, but they can potentiate the binding of 2-arachidonoyl-glycerol (Ben-Shabat et al (1998) Eur. J. Pharmacol. 353:23-31). Huestis provides another example of entourage effect and entourage compounds. Ethanolamines such as palmitoylethanolamine or stearoylethanolamine do not bind to CB1 or CB2 cannabinoid receptors, but they potentiate activity of ligands of cannabinoid receptors (Huestis (2002) Forensic Sci. Rev. 14:15-60). In another aspect, the present disclosure can exclude entourage compounds.

### EXAMPLES

### Example One

This is a study of odor perception, 10 mL of an odorant blend was added to two separate 20 mL vials. A small quantity of pigment was added to the second vial, but not the first. This resulted in two vials that differed only in a single variable: colored versus colorless. This was repeated for three other odorant blends using a different pigment each time. Pigments were matched to odorants based upon colors commonly associated with the odorant. This led to four sets of odorant vials, in which one vial of each odorant was colored and one was colorless with the following pigment/odorant combinations:

Orange (odorant)—Orange (pigment)

Lemon (odorant)—Yellow (pigment)

Lime (odorant)—Green (pigment)

Berry (odorant)—Blue (pigment)

Subjects were presented each, pair of odorant vials successively and asked to determine which of the pair smelled more intense. A choice of equal intensity was not an option. Subjects were told the name of the odorant/flavor. This was done for each pair of the four odorants and the results were recorded.

Table 1 provides Munsell data. The names orange, yellow, green, and blue, each refer to solutions of the present disclosure. The numbers on top (1, 2, 4, and 4) refer to solutions of the present disclosure. The numbers on top are lightest (1) to darkest (4) for each color. The data in the table refers to sixteen different solutions from the inventor's lab bench work. The designations in each of the cell of the table refer to specific Munsell codes. For example, “7.5Y 8/14” refers to one particular code from the Munsell book for the color of the pigmentation of each vial used.

Table of colored solutions of the present disclosure with corresponding Munsell codes.

TABLE 1
Colored solutions of the present disclosure with corresponding
Munsell color codes

| | | light . . . dark | | | | | | |
|---|---|---|---|---|---|---|---|---|
| | | 1 | 2 | | 3 | | 4 | |
| Orange | 1 | no color | 7.5Y | 8/14 | 10.0YR | 7/14 | 10.0R | 4/12 |
| Yellow | 2 | no color | 10.0Y | 9/2 | 10.0Y | 9/14 | 10.0Y | 8/12 |
| Green | 3 | no color | 10.0G | 8/2 | 2.5G | 7/8 | 2.5G | 3/12 |
| Blue | 4 | 7.5Y 8/14 | 10.0G | 8/4 | 5.0PB | 4/16 | 2.5PB | 1/6 |

Subject 1 (75% colored=more intense). Colored evaluated as more intense: Orange, Lemon, Berry; UN-colored more intense: Lime.

Subject 2 (75% colored=more intense). Colored evaluated as more intense: Orange, Lemon, Lime; UK-colored more intense: Berry.

Subject 3 (50% colored=more intense). Colored evaluated as more intense: Orange and Lemon. UN-colored more intense: Berry and Lime.

Odorants and associated solvents (or absence of solvents) are disclosed below. These odorants with solvents (where indicated) were used in the experiments described herein.

Orange: Orange Oil Terpeneless (no solvents); Vigon (504919) Lot #: 31025A03

Lemon: Lemon Oil Terpeneless (no solvents); Vigon (504922) Lot #: 28318A01.

Lime: Lime Oil Distilled Terpeneless (no solvents); Vigon (505005) Lot #: 31303A02.

Berry: Berry Mix Flavor (propylene glycol and ethanol base): The Flavor Apprentice Batch #: 16F1196.

The color of the completed solution that was used for testing subjects is disclosed below. Also disclosed below, is the color chart and number on the color chart that defines the color.

Orange: RAL 1033 49/22240.

Yellow: 16-202 John Deere Yellow (note: more fluorescent, i.e. yellow highlighter, than this shade).

Green: 40276 (American Colors Inc.)

Blue: 16-60 B&M Blue.

The number of milligrams of pigment added per 10 milliliters of solvent, for each of the blends, was as follows.

Orange: beta-carotene [CAS No. 7235-40-7], 1.0 mg/mL (i.e. 10 mg per 10 mL phytol). Supplier: Sigma (22040) Lot #: BCBL5379V.

Yellow: curcumin [CAS No. 458-37-7]]0.01 mg/mL (i.e. 0.1 mg per 10 mL phytol). Supplier: Sigma (C1386) Lot #: SLBN7214V.

Green: chlorophyllin Na Cu salt [CAS No. 11006-34-1], 1.0 mg/mL (i.e. 10 mg per 10 mL phytol). Supplier: Sigma (C6003) Lot SLBN2378V.

Blue: guiazulene [CAS No. 489-84-9], 0.1 mg/mL (i.e. 1.0 mg per 10 mL phytol). Supplier: Vigon International (504145) Lot #: 5055FA01.

### Example Two

This describes fee laboratory environment and study subjects. The approximate distance of the open vial from the subject's nose, when doing the perception experiment, was one to two inches. The duration of smelling time, when the subject was doing the perception experiment was one second. The delay in time, between the subject doing the smelling and the subject reporting the smell from one of the vials, was two seconds. The room used for testing was lit by a combination of incandescent light and natural light from a window during mid-afternoon. No fluorescent light was used. The noise level in the testing room was residential quiet. The study subjects were between the ages of about 35 to about 55.

### Example Three

Table 2 discloses reagents, compositions, and concentrations of the present disclosure.

TABLE 2
Exemplary reagents and formulations of the present disclosure

```
Sesquiterpenes (guaiene type)
    Guaiazulene
    Chamazulene
    Vetivazulene
    % Ranges
        1-50% guaiene type sesqui
        50-99% terpene blend/phytol
        0-1% guaiene type sesqui
        0-15% terpene blend
        0-95% diluent (dipropylene glycol/phytol)
        0-95% cannabinoids
Examples of formulas
    Cannabis ecig (e-cigarette) liquid
        0.5% guaiazulene
        10% terpenes
        65% phytol
        24.5% cannabinoids
    Colored concentrate
        0.01% chamazulene
        5% terpenes
        94.99% cannabinoids
    Colored ecig liquid
        0.01% vetivazulene
        10% terpene blend
        89.99% dipropylene glycol
Carotenoids
    Carotenes
        Lycopene (E160d)
        Beta-carotene (E160a) N
    Xanthophylls
        Violaxanthin N
        Neoxanthin N
        Bixins (E160b)
        Apocarotenal (E160e)
        Food orange 7(E160f)
        Flavoxanthin (E161a)
        Lutein (E161b) N
        Cryptoxanthin (E161c) N
        Rubixanthin (E161d)
        Violaxanthin (E161e)
        Rhodoxanthin (E161f)
        Canthazanthin (E161g)
        Zeaxanthin (E161h) N
        Citranaxanthin (E161i)
        Astaxanthin (E161j)
    Concentrated solutions
        1-5% carotenoid
        95-99% terpene blend/phytol/dipropylene glycol
    Final product ranges
        0-1% carotenoid
        0-15% terpene blend
        0-95% diluent
        0-95% cannabinoid
    Diarylheptanoids
        Curcumin (E100)
        Curcumenoids
    Concentrated solutions
        0-0.1% curcumin
        99.9-100% terpene blend/phytol/dipropylene glycol
    Final product ranges
        0.00001-0.1% curcumin
        0-15% terpene blend
        0-15% terpene blend
        0-95% diluent
        0-95% cannabinoids
Chlorophylls
    Chlorophyllin (E141)
    Concentrated solutions
        0-1% chlorophyllin
        99-100% dipropylene glycol
        0-15% terpene blend
        0-95% diluent
        0-95% cannabinoids
Flavonoids
    Anthocyanidins
    Anthocyanins (E163)
    Anthoxanthins
    Concentrated solutions
        1-10% flavinoid
        90-99% diluent
        0-1% flavinoid
        0-15% terpene blend
        0-95% diluent
        0-95% cannabinoids
Other
    Riboflavin (E101)
    Betanin (E162)
    Carmine
```

Plant pigments. This discloses the application of plant pigments to cannabis products. Pigments for use in compositions of the present, disclosure include the following. Guaiene sesquiterpenes, carotenoids, anthocyanins, anthocyanidins, and diarylheptanoids. Guaiane sesquiterpenes—guaiazulene and vetriazulene xanthophylls (bixins E160b, apocarotenal E160e, food orange 7 E160f, flavoxanthin E161a, lutein E161b cryptoxanthin E161c, rubixanthin E161d, violaxanthin E161e, rhodoxanthin E161f, canthaxanthin E161g, zeaxanthin E161f, citranaxanthin E161i, astaxanthin E161j) carotenes (lycopene E160d, beta-carotene E160a) diarylheptanoids (curcumin E100, curcumenoids) Anthocyanidins and anthocyanins betanin (E62) carmine chlorophyllin (E141) natural green 3.

Native Pigments. neoxanthin, violaxenthin, zeaxanthin, lutein, b-cryptoxanthin, b-carotene, chlorophyll a & b.

Colors of Pigments. Guaiazulene can be used to color a clear extract shades of blue or yellow extract shades of yellow-green. Carotene and lutein can be used to color extracts orange. Lycopene can be used to color extracts orange-red. Curcumins can be used to color an extract bright yellow and make them fluoresce under UV radiation. Guaiazulene and curcumins together can make shades of green that fluoresce. Anthocyanins can make extracts many shades of purple, red and blue.

Smell of Pigments. In a non-limiting embodiment, pigmentation of cannabis extracts does not lead to off flavors.

Solubilities of Pigments. The present disclosure encompasses solubility embodiments. Anthocyanins are only soluble in OBP (dipropylene glycol), not it nexus or cannabinoids alone. Anthocyanins in OBP can be added to cannabinoids and terpenes to make red colored extracts.

Guaiazulene is soluble in cannabinoids, terpenes, nexus, and obp and all useful combinations. Curcumins are soluble at necessary levels for full pigmentation of obp, nexus, and terpenes. Lutein and carotene are soluble in both obp, nexus, terpenes, and cannabinoids.

Without implying any limitation on the present disclosure, pigments that are non-plant pigments include hemoglobin, indigo carmine, allura red, quinoline yellow, FD&C Blue No. 1—Brilliant Blue FCF, FD&C Blue No. 2—Indigotine, FD&C Green No. 3—Fast Green FCF, FD&C Red No. 3—Erythrosine, FD&C Red No. 40—Allura Red AC, FD&C Yellow No. 5—Tartrazine, FD&C Yellow No. 6—Sunset Yellow FCF, Citrus Red 2 (orange shade), Orange B (red shade).

### Example Four

Examples of formulas are disclosed in Table 3.

TABLE 3
Examples of formulas

```
This formula encompasses products that for sale for the
consumer to add to their extracts.
    diluents 85-95%
    terpenes 5-15%
    pigments 0.0001-1%
This formula is a final diluted inhalable product.
    cannabinoids 10-50%
    diluents 45-80%
    terpenes 5-15%
    pigments 0.0001-1%
This formula is a final undiluted inhalable product
    cannibinoids 85-90%
    terpenes 5-10%
    pigments 0.0001-1%
This formula is an in-process ingredient used for
the dilution, flavoring, and coloring of extract
    diluents 50-99%
    terpenes 5-50%
    pigments 0.0001-50%
```

### Example Five

This disclosed the 10 terpenes that the present inventors report for each flavor in descending order of prominence.

Tangie (orange): D-Limonene, beta-caryophyllene, beta-myrcene, linalool, beta-pinene, alpha-humulene, alpha-bisabolol, alpha-pinene, terpinolene, caryophyllene-oxide [none] (Tangie contains all of these terpenes, but does not have any caryophyllene-oxide.)

Lemon Kush (yellow): D-Limonene, beta-pinene, beta-caryophyllene, beta-myrcene, linalool, alpha-humulene, alpha-bisabolol, alpha-pinene, terpinolene, caryophyllene-oxide [none] (Lemon Kush contains all of these terpenes, but does not have any caryophyllene-oxide.)

Lime Dream (green): D-Limonene, beta-myrcene, alpha-pinene, beta-caryophyllene, beta-pinene, alpha-humulene, linalool, alpha-bisabolol, caryophyllene-oxide, terpinolene.

Berry Cush (blue): Beta-myrcene, beta-caryophyllene, D-Limonene, alpha-pinene, alpha-humulene, beta-pinene, linalool, alpha-bisabolol, terpinolene, caryophyllene-oxide, terpinolene.

For the formulations named. Tangie, Lemon Kush, Lime Dream, and Berry Cush, these formulations are colorless. Except for alpha-bisabolol, the terpenes have a flavor. Alpha-bisabolol is the only terpene that when tasted by itself does not have a flavor.

### Example Six

In synesthesia embodiments, the present disclosure provides a pigmented oil with a color commonly associated with a particular odorant/flavor (e.g. green pigment with lime odorant/flavor) increases a users perception of the intensity, and possibly identity, of the lime flavor. This embodiment uses the sense of sight to make the vaporizable oil products taste better. In the conducted tests, the colors of the compositions were clearly evident to all subjects, and the investigators intentionally drew the attention of the study subjects to the colors. The compositions were in transparent glass vials.

The present disclosure provides a composition that comprises one or more of the above reagents. Also, the present disclosure provides a composition that consists of only one, only two, only three, only four, only five, only six, only seven, only eight, only nine, or only ten of the above reagents. Moreover, the present disclosure provides methods of manufacture, methods of laboratory testing with human subjects (testing for aesthetic appeal testing for preference, testing for medicinal qualities), and methods of use by a consumer, where the method uses one or more of the above reagents. Also, the method can exclude one or more of the above reagents.

The present disclosure provides tests for human subjects that can determine a composition that is most preferred and a composition that is least preferred from a predetermined selection of compounds. Also, the present disclosure can determine a composition that is preferred over all other compositions. The following is one practical test, useful with human subjects.

Please take a participant card and fill out the name, age and smoker-status sections. Next you will see four sets of four vials in front of you. Each is labeled with a number designation (e.g. 3.2). The first number designates the aroma and the second number is a variation of that aroma. For each aroma, smell all four variations and choose the one you find the MOST pleasing and the one you find the LEAST pleasing. Mark these selections on your participant card. Try nor to spill any liquid on you. If you do, simply wash with soap and water to remove. Vial contents are non-toxic, but may stain clothing. After evaluating all four aromas and making your selections on the participant card, feel free to leave any thoughts, or other utterances, in the comments section. Please fold and place your completed Participant Card into the accompanying Completed box and kindly refrain from discussing with others unless they have already taken part in the experiment. Your participation in the advancement of aroma perception science is greatly appreciated.

### Pigment, Color, Flavor, and Solubility Embodiments

Generally, the term pigment refers compositions that can be identified or distinguished by names of chemicals and where the pigment is perceivable as having a color. The term color refers to what is perceived by a human subject. A spectrophotometer can also determine color.

Potential Pigments: Guaiene sesquiterpenes, carotenoids, anthocyanins, anthocyanidins, and diarylheptanoids. Guaiane sesquiterpenes—guaiazulene and vetriazulene, xanthophylls (bixins E160b, apocarotenal E160e, food orange 7 E160f, flavoxanthin E161 a , lutein E161 b cryptoxanthin E161c, rubixanthin E161d. violaxanthin E161e, rhodoxanthin E161f, canthaxanthin E161g, zeaxanthin E161f, citranaxanthin E161i, astaxanthin E161j). carotenes (lycopene E160d, beta-carotene E160a) diarylheptanoids (curcumin E100, curcumenoids), Antbocyanidins and anthocyanins, betanin (E162), carmine, chlorophyllin (E141) natural green 3.

Native Pigments: neoxanthin, violaxenthin, zeaxanthin, lutein, b-cryptoxanthin, b-carotene, chlorophyll a & b, guaizulene.

Colors of Pigments: Guaiazulene can be used to color a clear extract shades of blue or yellow extract shades of yellow-green. Carotene and lutein can be used to color extracts orange. Lycopene can be used to color extracts orange-red. Curcumins can be used to color an extract bright yellow and make them fluoresce under UV radiation. Guaiazulene and curcumins together can make shades of green that fluoresce. Anthocyanins can make extracts many shades of purple, red and blue.

Smell of Pigments: The present disclosure demonstrates that the pigmentation of cannabis extracts does not lead to off flavors.

OFF-FLAVOR TESTING. Methods and equipment for detecting and quantifying off-flavors are available. See, for example. Marsili R G (1999) SPME-MS-MVA as an Electronic Nose for the Study of Off-Flavors in Milk. J. Agric. Food Chem., 47:648-654; Wilkes J G et al (2000) Sample preparation for the analysis of flavors and off-flavors in foods. Journal of Chromatography A. 880:3-33; Benanou D et al (2003) Analysis of off-flavors in the aquatic environment by stir bar sorptive extraction—thermal desorption—capillary GC/MS/olfactometry. Analytical Bioanalytical Chem. 376:69-77.

Solubilities of Pigments: Anthocyanins are only soluble in dipropylene glycol (i.e. OBP), not in nexus or cannabinoids alone. Anthocyanins in OBP can be added to cannabinoids and terpenes to make red colored extracts. Guaiazulene is soluble in cannabinoids, terpenes, nexus, and obp and all useful combinations. Curcumins are soluble at necessary levels for full pigmentation of obp, nexus, and terpenes. Lutein and carotene are soluble in obp, nexus, terpenes, and cannabinoids.

Non-Chemical Pigment Influences: Pigments may be modified by use of light, lightwaves, ultrasonic waves, other sound wave, and wave influences. LED lights on apparatuses that may contain the pigments or be in touch with the pigments, being soluble in obp, nexus, terpenes or cannabinoids.

Preference Testing by Human Subjects

The present disclosure provides, without implying any limitation, protocol for testing preferences by human subjects. The protocol can require that human subject designate either preferred or not preferred. Another embodiment for protocol, can require that the subject designate preference in terms of a scale, for example, a scale ranging from zero (least preferred) to ten (most preferred), where the scale takes the form of 0, 1, 2, 3, 4, 5, 6, 7, 8, 9, or 10. Also, the scale can range from zero to two (0, 1, 2), or zero to three (0, 1, 2, 3), or zero to four (0, 1, 2, 3, 4), and the like. The invention of the present disclosure can exclude any composition, or can exclude any method that uses a composition, where the composition was not determined to be preferred by at least 50% of a population of human subjects (where preference was determined under controlled laboratory testing conditions).

In preferred embodiments of the present composition and related methods, the present composition can require preference by human subjects in a controlled laboratory testing environment, that a given composition be preferred (in tests given only the option of preferred versus not preferred), by at least 20%, by at least 30%, by at least 40%, by at least 50%, by at least 60%, by at least 70%, by at least 80%, by at least 90%, or by at least 95% of the human subjects.

In preferred embodiments of the present composition and related methods, the present composition can require preference by human subjects in a controlled laboratory testing environment, that a given composition be preferred (in tests giving the options of zero (least preferred), one, and two (most preferred)), that the a preference value of “two” is designated by at least 20%, by at least 30%, by at least 40%, by at least 50%, by at least 60%, by at least 70%, by at least 80%, by at least 90%, or by at least 95% of the human subjects.

In preferred embodiments of the present composition and related methods, the present composition can require preference by human subjects in a controlled laboratory testing environment, that a given composition be preferred (in tests giving the options of zero (least preferred), one, two, and three (most preferred)), that the a preference value of “two” is designated by at least 20%, by at least 30%, by at least 40%, by at least 50%, by at least 60%, by at least 70%, by at least 80%, by at least 90%, or by at least 95% of the human subjects.

In preferred embodiments of the present composition and related methods, the present composition can require preference by human subjects in a controlled laboratory testing environment, that a given composition be preferred (in tests giving the options of zero (least preferred), one, two, and three (most preferred)), that the a preference value of “three” is designated by at least 20%, fay at least 30%, by at least 40%, by at least 50%, by at least 60%, by at least 70%, by at least 80%, by at least 90%, or by at least 95% of the human subjects. This criterion optionally can be combined with the criterion set forth in the previous paragraph.

What can be excluded is any composition or method, where the composition was not determined to be preferred by at least 60%, by at least 70%, by at least 80%, by at least 85%, by at least 90%, by at least 95%, by at least 99% of the human subjects. For these exclusionary embodiments, the term “preferred” can refer to tests that required either a yes/no preference level.

Alternatively, where tests required that subjects designate a preference on a scale of zero to three, or zero to four, or zero to five, and so on, what can be excluded are compositions and method, where at least 50% of the subjects did not designate at least a “two,” or where at least 50% of the subjects did not designate at least a “three,” or where at least 50% of the subjects did not designate at least a “four,” or where at least 50% of the subjects did not designate at least a “five,” or where at least 50% of the subjects did not designate at least a “six” or where at least 50% of the subjects did not designate at least a “seven,” or where at least 50% of the subjects did not designate at least an “eight,” and so on.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A formulation comprising a coloring and an odorant, wherein the odorant provides a perceivable odor to the formulation, and wherein the coloring provides a perceivable color to the formulation.

2. The formulation of claim 1, wherein in the coloring in pure form does not have a perceivable odor, and wherein the odorant in purified form does not have a perceivable color.

3. The formulation of claim 1, wherein the perceivable odor is orange, lemon, lime, or berry.

4. The formulation of claim 1, wherein the coloring is orange, yellow, green or blue.

5. The formulation of claim 1 that at room temperature and atmospheric pressure is one of a liquid, slurry, paste, oil. powdered solid, a non-powdered solid, or any combination thereof.

6. The formulation of claim 1, wherein the odorant in purified form has a first odor, wherein the odorant in purified form, mixed with a coloring in purified form has a second odor, and wherein the first odor is more intense than the second odor as determinable with perception tests with human subjects.

7. The formulation of claim 1, wherein the odorant in purified form has a first odor, wherein the odorant in purified form mixed with a coloring in purified form has a second odor, and wherein the first odor is less intense than the second odor as determinable with perception tests with human subjects.

8. The formulation of claim 1, wherein the odorant in purified form has first odor that is perceivable as smelling like a first kind of fruit, wherein the odorant in purified form mixed with a coloring in purified form has a second odor that is perceivable as smelling like a second kind of fruit.

9. The formulation of claim 1 that is capable of use in an electronic cigarette, wherein the electronic cigarette is capable in use of emitting a smoke, and wherein the coloring is capable of being perceived as having a color in the smoke emitted by the electronic cigarette.

10. The formulation of claim 1 that is capable of use in a beverage for human consumption.

11. The formulation of claim 1 that is capable of use in one or more of a skin lotion, perfume, cosmetic, for human consumption.

12. The formulation of claim 1 that is capable of use in a confection, cake frosting, or candy, for human consumption.

13. The formulation of claim 1 that comprises at least one plain pigment and at least one cannabinoid.

14. The formulation of claim 1 that comprises at least one plant pigment, and at least one terpene.

15. The formulation of claim 1 that comprises at least one non-plant pigment and at least one cannabinoid.

16. The formulation of claim 1 that comprises at least one non-plant pigment and at least one terpene.

17. The formulation of claim 1 that comprises D-Limonene, beta-caryophyllene, beta-myrcene, linalool, beta-pinene, alpha-humulene, alpha-bisabolol, alpha-pinene, terpinolene, but not any caryophyllene-oxide, optionally with orange pigment (Tangie).

18. The formulation of claim 1 that comprises D-Limonene, beta-pinene, beta-caryophyllene, beta-myrcene, linalool, alpha-humulene, alpha-bisabolol, alpha-pinene, terpinolene, but not any caryaphyllene-oxide, optionally with yellow pigment (Lemon Rush).

19. The formulation of claim 1 that comprises D-Limonene, beta-myrcene, alpha-pinene, beta-caryophyllene, beta-pinene, alpha-humulene, linalool, alpha-bisabolol, caryophyllene-oxide, terpinolene, optionally with green pigment (Lime Dream).

20. The formulation of claim 1 that comprises Beta-myrcene, beta-caryophyllene, D-Limonene, alpha-pinene, alpha-humulene, beta-pinene, linalool, alpha-bisabolol, terpinolene, caryophyllene-oxide, terpinolene, optionally with blue pigment (Berry Cush).

21. The formulation of claim 17, comprising orange pigment, wherein the orange pigment acts by synesthesia to increase preference by a human subject of one or both of the odor or flavor of Tangie.

22. The formulation of claim 18, comprising yellow pigment, wherein the yellow pigment acts by synesthesia to increase preference by a human subjects of one or both of the odor or flavor of Lemon Kush.

23. The formulation of claim 19, comprising green pigment, wherein the green pigment acts by synesthesia to increase preference by a human subject of one or both of the odor or flavor of Lime Dream.

24. The formulation of claim 20, comprising blue pigment, wherein the blue pigment acts by synesthesia to increase preference by a human subject of one or both of the odor or flavor of Berry Cush.

25. A formulation for an electronic cigarette comprising 0.05%-5% guaiazulene, about 10% terpenes, about 65% phytol, and about 24.5% cannabinoids, wherein the perception of grape odor is greater than that for a formulation comprising 0-0.03% guaiazulene, about 10% terpenes, about 65% phytol, and about 24.5% cannabinoids.

26. A formulation for an electronic cigarette comprising 0.05%-5% guaiazulene, about 10% terpenes, about 65% phytol, and about 24.5% cannabinoids, wherein the perception of grape flavor is greater than that for a formulation comprising 0-0.03% guaiazulene, about 10% terpenes, about 65% phytol, and about 24.5% cannabinoids.

27. A formulation for an electronic cigarette, comprising 0.01%-0.05% chamazulene, about 5% terpenes, and about 94.99% cannabinoids, wherein the perception of grape odor is greater than that for a formulation comprising 0-0.005% chamazulene, about 5% terpenes, and about 94.99% cannabinoids.

28. A formulation for an electronic cigarette, comprising 0.01%-0.5% chamazulene, about 5% terpenes, and about 94.99% cannabinoids, wherein the perception of grape flavor is greater than that for a formulation comprising 0-0.005% chamazulene, about 5% terpenes, and about 94.99% cannabinoids.

29. A formulation of claim 1 contained within a vapor cartridge of an electronic cigarette that comprises a light emitting diode.
